(19) United States
(12) Patent Application Publication
(10) Pub. No.: US 2002/0068299 A1
(43) Pub. Date: Jun. 6, 2002

(54) High-density microarrays

(76) Inventor: Rajan Kumar, Robbinsville, NJ (US)

Correspondence Address: Rajan Kumar, 18 Buford Road, Robbinsville, NJ, 08691, US

(21) Appl. No.: 10/010,684

(22) Filed: Dec. 5, 2001

Related U.S. Application Data
(60) Provisional application No. 60/251,332, filed on Dec. 6, 2000.
(60) Provisional application No. 60/268,132, filed on Feb. 13, 2001.

(51) Int. Cl. C12Q 1/68; G01N 33/53; G01N 33/542; C12M 1/34

(52) U.S. Cl. 435/6; 435/7.9; 435/287.2

(57) ABSTRACT

The invention describes methods to fabricate, use and analyze three-dimensional DNA microarrays. Such microarrays are used for investigation of gene expression profiles. The three-dimensional microarrays have many advantages over the current microarray technologies, including a higher effective probe density.

### CROSS-REFERENCE TO RELATED APPLICATIONS

This application claims the benefit under 35 U.S.C. 119(e) U.S. Provisional Application 60/251,332, filed Dec. 6, 2000, and U.S. Provisional Application 60/268,132, filed Feb. 13, 2001. This application is related to U.S. patent applications entitled “Fluidic Arrays” filed Oct. 25, 2001 (Docket No. GDS_NP_{—}2001_{—}002) and “Stacked Arrays”, filed Oct. 29, 2001 (Docket No. GDS NP_{—}2001_{—}001). These applications are incorporated herein by reference.

### STATEMENT REGARDING FEDERALLY SPONSORED RESEARCH OR DEVELOPMENT

No Government License Rights

### BACKGROUND OF THE INVENTION

The present invention lies in the field of molecular biology and is particularly concerned with the technique of microarrays used for detection of molecules of interest in a sample, determination of composition of a complex mixture of molecules, and comparison of composition of two or more samples of molecules, such molecules including although not exclusively, DNA, RNA and proteins.

Sequencing of a large number of genomes has generated a growing body of DNA sequence information that promises to revolutionize experimental design and data interpretation in pursuit of biological understanding. However, collection of sequence data, by itself, is not sufficient to decipher the roles of genes and gene products in cellular and organismal function. Therefore, there has been a concomitant growth in development of technologies to exploit the massive amount of DNA sequence data.

One of such revolutionary technologies to emerge in the biotechnology area is the microarray technology. Microarrays, consisting of high-density arrangements of oligonucleotides or complementary DNAs (cDNAs) can be used to interrogate complex mixtures of molecules in a parallel and quantitative manner. When a sample analyzed by microarray technology is derived from a population of mRNA of a cell or cell population, the analysis provides information about the genes that are present in that cell or cell population. Similarly, arrays of proteins, peptides and other small molecules are also being fabricated for analysis of samples for protein-protein interactions, protein-DNA interactions, protein function, and drug discovery. The applications of microarrays include diagnostic and environmental testing, genomic research at academic institutions, biotechnology and pharmaceutical companies, and drug discovery.

The procedure to use microarrays is described here with reference to use of DNA microarrays. DNA microarrays are used to measure concentrations of nucleic acid populations in a sample by hybridization. Typically, a large number of DNA fragments (called probes) are attached to a solid substrate to create an array. Each probe is attached to a defined place. The nucleic acids in the sample (called targets) are labeled usually with fluorescent dyes, typically fluorescein, Cy3 and/or Cy5, or with radioactive labels such as phosphorus 33 or sulfur 35. When the array of probes is exposed to the sample, the target nucleic acids in the sample hybridize to specific probes on the array. By shining light of appropriate wavelength, the array is then visualized to determine which probes are hybridized thereby giving an estimate of the nucleic acids present in the sample.

Typically, microarrays are generated on glass substrates, usually I mm thick slides, with a size of 1 inch by 3 inches. The microarrays are created by depositing molecules of interest in defined locations on one surface of the glass substrate. One of the limitations of such arrays is that the number of molecular species that can be included on an array is limited by the amount of surface area available. To increase the number of molecular species that can be deposited on an array surface, and therefore, can be used to simultaneously interrogate a sample, the size of the elements has to be reduced. Such reduction in the size of individual elements has an effect of reducing the sensitivity of detection of interactions between array elements and sample constituents. Therefore, there is a need for innovative approaches that can increase the number of molecular species in an array without reducing the size of individual elements.

Currently, there are two different technologies established to make microarrays—in situ synthesis method; and Deposition of pre-synthesized DNA.

The two methods differ in the length of the probes deposited. In situ synthesis methods typically use small-length probes due to complexity of individual synthesis steps. For example, the Affymetrix microarrays usually consist of 20-mer probes. The deposition of presynthesized DNA can involve longer probes, even complete cDNAs (complementary DNAs that are made from reverse transcription of the messenger RNAs present in the cell). Alternatively, the Polymerase Chain Reaction products can be used as probes. The limitations of current technologies include high cost of manufacture, low resolution and sensitivity, lack of customization, low array density, and requirement of specialized and expensive instrumentation.

A method for fabricating microarrays, of biological samples has been described (see Brown et. al., U.S. Pat. No. 5,807,522). The method involves dispensing a known volume of reagent at each selected array position, by tapping a capillary dispenser on the support under conditions effective to draw a defined volume of liquid onto support. The method can be used to dispense distinct nucleic acids in discrete spots and therefore, to create microarrays of about 100 or about 1000 spots per 1 square centimeters. Each spot is created by dispensing a volume of liquid between 0.002 and 0.25 nl.

Heyneker (U.S. Pat No. 6,067,100) teach another method for fabricating arrays of oligonucleotides comprising a solid substrate comprising a plurality of different oligonucleotide pools, each oligonucleotide pool arranged in a distinct linear row to form an immobilized oligonucleotide stripe, wherein the length of each stripe is greater than its width. The oligonucleotides are attached to the solid matrix covalently. Alternatively, each oligonucleotide species is attached to fibers individually and then assembled into a strip on a solid support. Such strips from multiple oligonucleotide pools can be arranged side to side on a solid support to obtain a composite array. The presence of a solid support backing, which preferably is plastic, is always necessary and the use of these arrays in the absence of a solid support is not contemplated.

Walt et al (U.S. Pat No. 5,244,636) describe a fiber optic sensor which is able to conduct multiple assays and analysis concurrently using molecules immobilized at individual spatial positions on the surface of one of the ends of the optical fiber bundle. The fiber optic bundle can be used to transmit excitation light of suitable wavelength to the molecules at the optical fiber end and also for transmission of the emission light back for detection. An array of oligonucleotides or peptides or any other molecules can be created on the ends of optical fibers and used as a microarray.

Multiple uses of microarrays have been described. One of the primary applications is determination of the nucleic acid or protein composition of a sample. Fodor et al (U.S. Pat No. 5,800,992) detail a method to compare the composition of two or more samples by labeling members of each of the samples with a distinct labeling molecule, preferably fluorescent molecules. The microarrays described by Fodor et al have at least 1,000 distinct polynucleotides per cm².

A use of protein microarrays has been described by MacBeath et. al. Miniaturized assays were developed that accommodate extremely low sample volumes and enable the rapid, simultaneous processing of thousands of proteins. A high-precision robot was used to spot proteins onto chemically derivatized glass slides at high spatial densities. The proteins attached covalently to the slide surface yet retained their ability to interact specifically with other proteins, or with small molecules, in solution. Three applications for the protein microarrays thus generated were described: screening for protein-protein interactions, identifying the substrates of protein kinases, and identifying the protein targets of small molecules.

It is, therefore, an object of the present invention to provide improved fluidic methods and devices for analysis of samples using molecular arrays.

### BRIEF SUMMARY OF THE INVENTION

In general the invention involves molecular arrays on substrates such that the array elements are arranged in three space dimensions.

It is another object of the present invention to provide methods to make such arrays.

It is another object of the present invention to provide methods to use such arrays for analysis of samples.

It is further an object of the invention to provide methods for analysis of samples, which are fluorescently or radioactively labeled.

### BRIEF DESCRIPTION OF THE SEVERAL VIEWS OF THE DRAWING

FIG. 1 is a view of one embodiment of the invention that consists of a three-dimensional arrangement of array elements carried on glass tubes. Shown in the picture is an 8×8×5 (320 element) array of the invention enclosed in a hybridization chamber.

FIG. 2 shows the top view of the array of the invention shown in FIG. 1.

FIG. 3 shows the front view of the array of the invention shown in FIG. 1.

FIG. 4 shows the side view of the array of the invention shown in FIG. 1.

FIG. 5 shows a front view of a two-dimensional array that is used to assemble a three-dimensional array of the invention shown in FIG. 1.

### DETAILED DESCRIPTION OF THE INVENTION

Before providing a detailed description of the inventions of this patent, particular terms used in the patent will be defined.

An “array” is a device comprising a substrate that contains on its surface distinct spots or deposits of one or more than one molecular species. An example of an array in common use is the DNA microarray.

An “element” of an array is a distinct spot or deposition of molecules in a spatially localized area on the substrate of the array. Usually, an array element contains deposition of molecules of one particular species or sequence.

“Hybridization” is the process by which two strands of DNA or RNA come together to form a double-stranded molecule. For hybridization between two strands to take place, the sequence of the two strands must be completely or nearly so complementary.

“Complementary” strand of a given strand is a strand of DNA or RNA that is able to hybridize to the given strand and is characterized by the presence of nucleotides A, C, G, and T, respectively opposite to nucleotides T, G, C, and A, respectively, on the given strand.

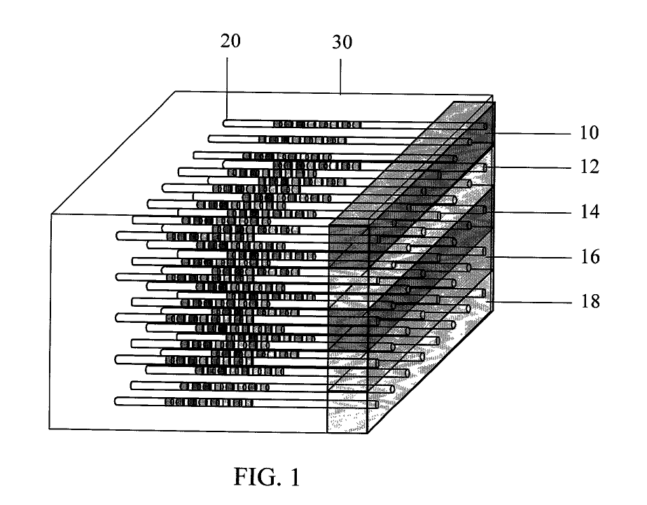

The arrays of the present invention are described with reference to FIG. 1. The three-dimensional array of the present invention comprises five two-dimensional arrays 10, 12, 14, 16, and 18 joined together on one end. Each of the five arrays comprises eight substrates. One of the substrates 20 that comprises two-dimensional array 10 is shown. Each of the eight substrates further comprises eight elements. The array of the invention shown in FIG. 1 comprises 320 elements. The molecules present in different elements can be similar or different.

As described in the previous application entitled “Fluidic Arrays” filed Oct. 25, 2001 (Docket No. GDS_NP_{—}2001_{—}002), the substrates can any of the various cross-sections. The substrate used can have either a solid core or a hollow core, or square, rectangular, circular or hexagonal cross-section. Additionally, the molecular deposition can the whole circumference of the substrate cross-section or partly. In addition, a molecular deposition can consist of different molecules on the different faces of the substrate. It will be obvious to anyone skilled in the art that when substrates with other cross-sections are used, the above principles of circumferential coating or partial circumference coating or coating with different material depositions can be employed.

The cross-sectional dimensions of the substrates will be between 1 micrometer and 10 centimeters, preferably between 10 micrometer and 10 millimeters. The length of the substrates is between 100 microns and 10 centimeter, preferably between 1 centimeter and 5 centimeter. The size of the elements on the substrate is between 10 micrometers and 1 millimeter. The shape of the elements on the substrate could be round, square, oval, irregular or any other shape.

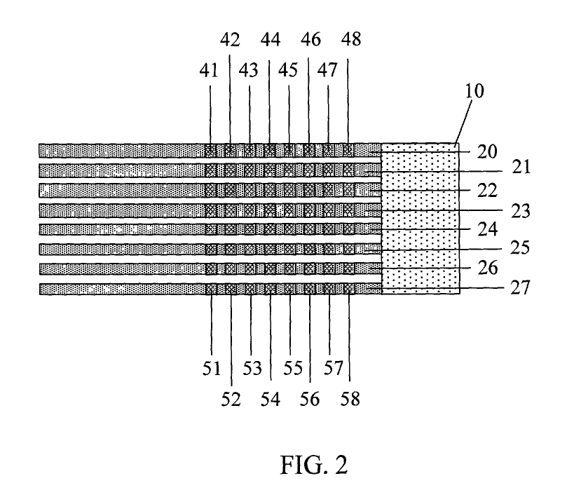

FIG. 2 shows the top view of the array of the invention. Only the array 10 is visible. Array 10 consists of eight substrates 20, 21, 22, 23, 24, 25, 26, and 27. Each of the substrates carries elements with different molecular depositions. For example, the substrate 20 carries elements 41, 42, 43, 44, 45, 46, 47, and 48 and substrate 27 carries elements 51, 52, 53, 54, 55, 56, 57, and 58.

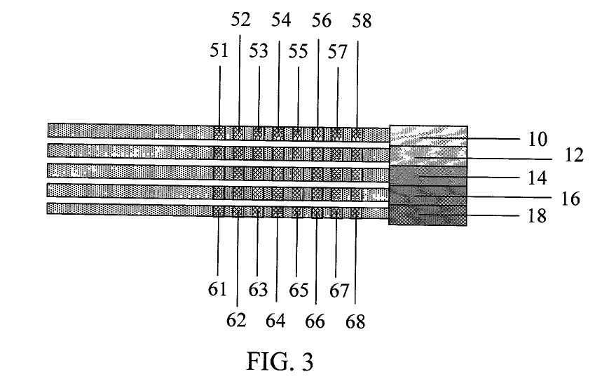

A front view of the array of the invention is shown in FIG. 3. The array consists of two-dimensional arrays 10, 12, 14, 16, and 18. The elements carried on each of the two-dimensional array are different. For example, the proximal substrate on array 10 carries elements 51, 52, 53, 54, 55, 56, 57, and 58, and the proximal substrate on the array 18 carries elements 61, 62, 63, 64, 65, 66, 67, and 68.

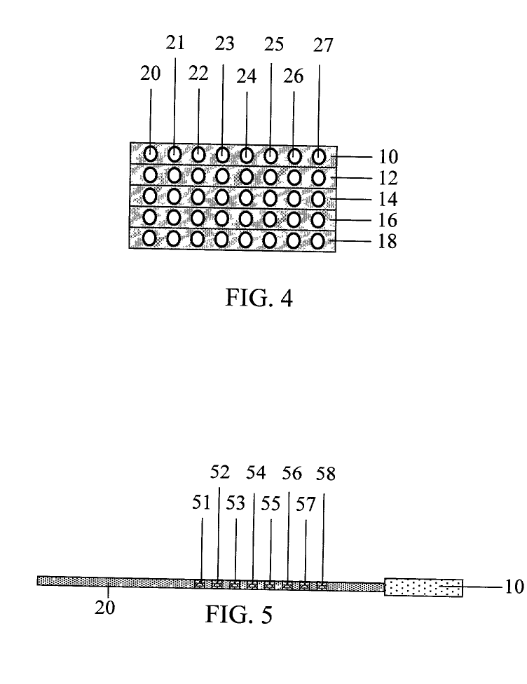

FIG. 4 shows the side view of the array of the invention. In this embodiment, the substrates extend to the end of the edge piece and are therefore, visible. Alternatively, the substrates might not extend to the end of the edge piece and not be visible. The array consists of two-dimensional arrays 10, 12, 14, 16, and 18. Each of the two-dimensional array consists of eight substrates. For example, array 10 consists of substrates 20, 21, 22, 23, 24, 25, 26, and 27.

FIG. 5 shows the side view of one of the two-dimensional arrays. The array 10 comprises eight substrates, but only one, substrate 20 is visible in this view. The substrate 20 carries elements 51, 52, 53, 54, 55, 56, 57, and 58. Although the figure shows that the substrates are held together on one end, other embodiments of the arrays can have the substrates held together on both ends.

In another embodiment, to generate the two-dimensional arrays, molecular depositions are made on a thin substrate e.g. 150-micron glass or plastic. The substrate material in between the molecular depositions is removed to one edge of the substrate, leaving the areas of molecular depositions held together by the other edge of the substrate. Such removal of the substrate can occur either before or after the depositions. Glass sheets in the thickness of 50 micrometer are commercially available and can be used for this purpose. Alternatively, plastic sheets with thickness as little as 10 microns or less can be used. To increase the firmness of plastic substrate, it can be supported with glass or metal inserts.

One of the advantages of these arrays is that the target molecules are able to diffuse faster between different locations and reach the corresponding probe. Another advantage of the present arrays is that amount of surface area available for spotting is larger than conventional arrays and therefore, a larger number of probes can be exposed to the targets in the sample simultaneously.

The linear depositions of functionalization can be made on the substrate using any of a number of methods. The functionalization can be performed by drawing using rollers, pens or quills or by printing using inkjet or bubble jet printers. Additionally for polymeric biological molecules such as DNA, proteins and RNA, the appropriate functionalization can be added to the fiber using in situ synthesis using photolithography or ink jet printing.

The molecules that are deposited on the substrates are usually covalently coupled to the substrate material. The choice of a particular method for coupling specific molecules to a substrate depends on characteristics of the molecules and the substrate. For example, a number of methods are known in the art for coupling DNA molecules to glass substrates, including coupling of amino-terminated nucleotides to aldehyde coated glass substrates. Similarly, a number of methods for coupling protein molecules to plastic substrates are known in the art, and can be used to create the arrays of the present invention.

In another embodiment, the elements of the array are created on both surfaces of a substrate. The arrays on the two surfaces of a substrate can consist of the identical spots or different spots. If the array on the two surfaces consist of identical spots, they can be detected simultaneously or separately. The advantage of simultaneous detection is higher sensitivity; the advantage of having different spots and separate detection is increase in density of elements of the array.

The detection of products captured on the elements of the array can be done by a number of detection techniques. The products captured on the elements can be studied in situ with fluorescence or by selective release from the fiber. Or the arrays can be analyzed by other biophysical techniques such as mass spectrometry after release of the product.

One particular use of the arrays of invention is analysis of DNA or RNA samples by hybridization. Another use is to study interaction of proteins with DNA or with other proteins or small molecules e.g. antibody-antigen interactions.

The deposition of the molecules on the substrate can be performed by drawing using rollers, pens or quills. Additionally for polymeric biological molecules such as DNA, proteins and RNA, the appropriate deposition can be performed on the substrate using in situ synthesis, e.g. using photolithography or ink jet printing. Multiple fibers can be laid parallel to each other for the deposition process.

The arrays of the invention can also be combined with molecular biology reagents and instructions to design kits for genomic and proteomic research as well as for drug discovery.

Although the invention has been described in some detail by way of illustration and example for purposes of clarity and understanding, it may be readily apparent to those of ordinary skill in the art in light of the teachings of this invention that certain changes and modifications may be made without departing from the spirit or scope of the appended claims.

### EXAMPLE 1

Deposition of Molecular Elements on a Linear Array

Take a square cross-section borosilicate glass tube with each side measuring 330 microns and use them for creating the substrate. Attach amino functional groups to the surface of the substrate by treating it with N-(2-Aminoethyl)-3-aminopropyltrimethoxysilane. Spot human cDNA molecules of interest on the substrate using a felt-tip pen. Allow the cDNA molecules to attach to the amino groups and wash. Dry the substrates. The arrays are now ready for use.

### EXAMPLE 2

Creating a Two-dimensional Array by Assembly of Substrates

Take four square cross-section borosilicate glass tube, 20 mm long, with each side measuring 330 microns and treat them for attaching the amino functional group as in example 1. Place them parallel to each other in a fixture at a spacing of 330 microns. Make sure that the substrates extend 5 mm beyond the fixture at one of their ends. Using a felt tip pen, draw lines with eight human cDNA samples across the substrates. Take two pieces of polycarbonate, 10 mm square, to use as edge pieces. Machine four grooves in each of them at a spacing of 330 microns, each groove measuring 330 microns wide and 165 micron deep. Align the ends of the four arrays extending beyond the fixture with the four grooves in the edge pieces and bond the edge pieces together, holding the arrays together. Now repeat the process for another seven groups of four tube substrates each, using a different set of human cDNA samples for each group. Stack the eight arrays so generated with their polycarbonate molds on one end. Using four molds, attach the other ends of the eight arrays with epoxy such that one substrate from each of the eight groups is present in each mold. Allow the epoxy to solidify. Cut the glass substrates near the polycarbonate to generate four arrays of eight tube substrates each. Each of the arrays will carry 64 different array elements.

### EXAMPLE 3

Fabrication of a Three-dimensional Array of the Invention

Follow the process described in example 2 to fabricate five two-dimensional arrays, each of the two-dimensional array carrying sixty four array elements made with different human cDNA samples. Align the epoxy ends of all five arrays together, and reversibly immobilize them. The process results in a three-dimensional array as shown in FIG. 1, containing 320 array elements.

### EXAMPLE 4

Analysis of a DNA Sample Using the Array of the Invention

Make a three-dimensional human cDNA array as described in example 3. Take a DNA sample of interest and label the DNA molecules present in the sample with Cy3. Add the fluorescently labeled sample into a container and dip the array in the sample so that all the array elements are immersed in the sample. Let the target molecules in the sample hybridize to the probes for 1hour. Take the array out and wash with 0.1 mM TE buffer (10 mM Tris HCl, 0.5 mM EDTA). At this point, separate the five two-dimensional arrays for detection. Position the first array under a fluorescent microscope equipped with a digital camera. Use an excitation light of 550 nm wavelength and observe and record the light intensity from each element at 570 nm emission wavelength. If the sample contains targets that complementary to the probes on the array, the light intensity recorded from the corresponding element(s) will be stronger than others. Repeat the process with the other four two-dimensional arrays.

What I claim as my invention is

1. An array of molecules deposited on one or more substrates such that the array elements are distributed in three space dimensions.

2. An array of claim 1 where the molecules are DNA, RNA or proteins.

3. A method for analysis of samples using an array of claim 1.

4. An array of claim 1 where the substrates used for deposition of array elements are made of glass.

5. An array of claim 1 where the substrates used for deposition of array elements are glass tubes.

6. An array of claim 1 where the cross-section of the substrates is less than 1 mm.

7. An array of claim 1 where the substrates used for deposition of array elements have a square cross-section.

8. An array of claim 1 comprising the array is fabricated from assembly of two-dimensional arrays, each of the two-dimensional array being different from other such arrays being assembled.

9. A method of claim 3 where the samples being analyzed are fluorescently labeled.

10. A method of claim 3 where the samples being analyzed are radioactively labeled.
